(19) United States
(12) Patent Application Publication
(10) Pub. No.: US 2016/0325095 A1
(43) Pub. Date: Nov. 10, 2016

(54) Novel Recording Approach of Stapedius Muscle Activity

(71) Applicant: MED-EL Elektromedizinische Geraete GmbH, Innsbruck (AT)

(72) Inventors: Christian Denk, Innsbruck (AT); Matthias Ladurner, Trins (AT); Markus Oberparleiter, Rum (AT)

(21) Appl. No.: 15/147,935

(22) Filed: May 6, 2016

Related U.S. Application Data
(60) Provisional application No. 62/157,505, filed on May 6, 2015.

(51) Int. Cl. A61N 1/36; A61N 1/05; A61B 5/00; A61B 5/0492; A61B 5/03

(52) U.S. Cl. CPC A61N 1/36032; A61B 5/0492; A61B 5/03; A61B 5/6867; A61N 1/0541

(57) ABSTRACT

A method of placing a stapedius activity sensor in a stapedius muscle of a patient is described. The stapedius muscle has a tendon end connecting to the stapes bone, and an opposing muscle belly end where the facial nerve innervates the stapedius muscle. A mastoidectomy is performed to create an opening through mastoid bone of the patient. A lead groove is drilled in temporal bone of the patient following a route along the facial nerve to the muscle belly end of the stapedius muscle. The stapedius activity sensor is then introduced along the lead groove to insert a distal end of the stapedius activity sensor into the muscle belly end of the stapedius muscle.

This application claims priority from U.S. Provisional Patent Application 62/157,505, filed May 6, 2015, which is incorporated herein by reference in its entirety.

### TECHNICAL FIELD

The present invention relates to hearing prosthesis systems such as cochlear implant systems, and more specifically to measurement of stapedius muscle activity for such systems.

### BACKGROUND ART

Most sounds are transmitted in a normal ear as shown in FIG. 1 through the outer ear 101 to the tympanic membrane (eardrum) 102, which moves the bones of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Hearing is impaired when there are problems in the ability to transduce external sounds into meaningful action potentials along the neural substrate of the cochlea 104. To improve impaired hearing, auditory prostheses have been developed. For example, when the impairment is associated with the cochlea 104, a cochlear implant with an implanted stimulation electrode can electrically stimulate auditory nerve tissue with small currents delivered by multiple electrode contacts distributed along the electrode.

FIG. 1 also shows some components of a typical cochlear implant system which includes an external microphone that provides an audio signal input to an external signal processor 111 where various signal processing schemes can be implemented. The processed signal is then converted into a digital data format, such as a sequence of data frames, for transmission into the implant 108. Besides receiving the processed audio information, the implant 108 also performs additional signal processing such as error correction, pulse formation, etc., and produces a stimulation pattern (based on the extracted audio information) that is sent through an electrode lead 109 to an implanted electrode array 110. Typically, this electrode array 110 includes multiple electrodes on its surface that provide selective stimulation of the cochlea 104.

Following surgical implantation, the cochlear implant (CI) must be custom fit to optimize its operation with the specific patient user. For the fitting process, it is important to know if an audible percept is elicited and how loud the percept is. Normally this information is gained using behavioral measures. For example, for each electrode contact the CI user is asked at what stimulation level the first audible percept is perceived (hearing threshold (THR)) and at what stimulation level the percept is too loud (maximum comfort level (MCL)). For CI users with limited auditory experiences or insufficient communication abilities (e.g., small children), these fitting parameters can be determined using objective measures.

FIG. 2 shows a portion of the middle ear anatomy in greater detail, including the incus 201 and the stapes 202. The lenticular process end of the incus 201 vibrates the head 205 of the stapes 202, which in turn vibrates the base 203 of the stapes 202 which couples the vibration into the inner ear (cochlea). Also connected to the head 205 of the stapes 202 is the stapedial tendon 206 of the stapedius muscle situated within the bone of the pyramidal eminence 207. When a loud noise produces an excessively high sound pressure that could damage the inner ear, the stapedius muscle reflexively contracts to decrease the mechanical coupling of the incus 201 to the stapes 202 (and thereby also reduce the force transmission). This protects the inner ear from excessively high sound pressures.

The tensing of the stapedius muscle when triggered by such high sound pressures is also referred to as the stapedius reflex. Medically relevant information about the functional capability of the ear may be obtained by observation of the stapedius reflex. Measurement of the stapedius reflex also is useful for setting and/or calibrating cochlear implants because the threshold of the stapedius reflex is closely correlated to the psychophysical perception of comfortable loudness, the so-called maximal comfort level (MCL). The stapedius reflex can be determined in an ambulatory clinical setting using an additional device, an acoustic tympanometer that measures the changes in acoustic impedance of the middle ear caused by stapedial muscle contraction in response to loud sounds.

To measure the stapedius reflex intra-operatively, it is known to use electrodes that are brought into contact with the stapedius muscle to relay to a measuring device the action current and/or action potentials generated, e.g. a measured EMG signal, upon a contraction of the stapedius muscle. But a reliable minimally-invasive contact of the stapedius muscle is difficult because the stapedius muscle is situated inside the bony pyramidal eminence and only the stapedial tendon is accessible from the interior volume of the middle ear.

Various intraoperative stapedius muscle electrodes are known from U.S. Pat. No. 6,208,882 (incorporated herein by reference in its entirety), however, these only achieve inadequate contact of the stapedius muscle tissue (in particular upon muscle contraction) and are also very traumatizing. This reference describes one embodiment that uses a ball shape monopolar electrode contact with a simple wire attached to it. That would be very difficult to surgically position into a desired position with respect to the stapedius tissue and to fix it there allowing for a long-term atraumatic and stable positioning. Therefore the weakness of this type of electrode is that it does not qualify for chronic implantation. In addition, there is no teaching of how to implement such an arrangement with a bipolar electrode with electrode contacts with sufficient space between each other to enable bipolar registration.

Some intraoperative experiments and studies have been conducted with hook electrodes that have been attached at the stapedius tendon or muscle. These electrode designs were only suitable for acute intra-operative tests. Moreover, some single hook electrodes do not allow a quick and easy placement at the stapedius tendon and muscle—the electrode has to be hand held during intra-operative measurements, while other double hook electrodes do not ensure that both electrodes are inserted into the stapedius muscle due to the small dimensions of the muscle and the flexibility of the electrode tips. One weakness of these intraoperative electrodes is that they do not qualify for chronic implantation.

German patent DE 10 2007 026 645 (incorporated herein by reference in its entirety) discloses a two-part bipolar electrode configuration where a first electrode is pushed onto the stapedius tendon or onto the stapedius muscle itself, and a second electrode is pierced through the first electrode into the stapedius muscle. One disadvantage of the described solution is its rather complicated handling in the very limited space of the surgical operation area, especially manipulation of the fixation electrode. In addition, the piercing depth of the second electrode is not controlled so that trauma can also occur with this approach. Also it is not easy to avoid galvanic contact between both electrodes.

U.S. Patent Publication 20100268054 (incorporated herein by reference in its entirety) describes a different stapedius electrode arrangement having a long support electrode with a base end and a tip for insertion into the target tissue. A fixation electrode also has a base end and a tip at an angle to the electrode body. The tip of the fixation electrode passes perpendicularly through an electrode opening in the support electrode so that the tips of the support and fixation electrodes penetrate into the target tissue so that at least one of the electrodes senses electrical activity in the target stapedius tissue. The disadvantages of this design are analogous to the disadvantages mentioned in the preceding patent.

U.S. Patent Publication 20130281812 (incorporated herein by reference in its entirety) describes a double tile stapedial electrode for bipolar recording. The electrode is configured to be placed over the stapedius tendon and a sharp tip pierces through the bony channel towards the stapedius muscle. The downside of this disclosure is again its rather complicated handling in the very limited space of the surgical operation area,

Various other stapedial electrode designs also are known, all with various associated drawbacks; for example, US 2011/0255731, US 2014/0100471, U.S. Pat. No. 8,280,480, and U.S. Pat. No. 8,521,250, all incorporated herein by reference in their entireties. A simple wire and ball contact electrode is very difficult to surgically position and to keep it atraumatically stabilized for chronic implantations. The penetrating tip of such a design must be stiff enough to pass through the bone tunnel, but if the tip is too stiff, it is difficult to bend and maneuver the wire into its position. And some stapedius muscle electrode designs are only monopolar electrodes (with a single electrode contact) and are not suitable for a bipolar arrangement with the electrode contacts with sufficient distance between each other to enable bipolar registration. Finally, another design is disclosed in co-pending U.S. Provisional Patent Application 62/105,260 (incorporated herein by reference in its entirety).

### SUMMARY

Embodiments of the present invention are directed to a method of placing a stapedius activity sensor in a stapedius muscle of a patient. The stapedius muscle has a tendon end connecting to the stapes bone, and an opposing muscle belly end where the facial nerve innervates the stapedius muscle. A mastoidectomy is performed to create an opening through mastoid bone of the patient. A lead groove is drilled in temporal bone of the patient following a route along the facial nerve to the muscle belly end of the stapedius muscle. The stapedius activity sensor is then introduced along the lead groove to insert a distal end of the stapedius activity sensor into the muscle belly end of the stapedius muscle.

In further specific embodiments, the method may further include implanting a stimulation element for a hearing implant into the patient, either before or after the stapedius activity sensor is introduced. In addition or alternatively, the method may also include providing test stimulus signals to the facial nerve and the stapedius muscle after drilling the lead groove to confirm proper continuing functioning of the facial nerve and the stapedius muscle. One or more of the steps may be performed by robotic surgery.

The stapedius activity sensor may be a pressure sensor or a sensing electrode such as a 1-3 French diameter electrode, an electromyography (EMG) recording electrode, and/or a bipolar electrode contact. The sensing electrode may include one or more connecting tines configured to secure the sensing electrode in a fixed position within the stapedius muscle when the distal end of the stapedius activity sensor is inserted into the muscle belly end of the stapedius muscle.

Embodiments of the present invention also include a hearing implant fitting system and/or a hearing implant system (e.g., a cochlear implant, auditory brainstem implant, or middle ear implant) having a stapedius activity sensor inserted according to any of the foregoing.

### BRIEF DESCRIPTION OF THE DRAWINGS

FIG. 1 shows anatomical structures of a human ear having a cochlear implant system.

FIG. 2 shows detailed anatomy around the stapedius tendon in a human ear.

FIG. 3 shows the anatomy of the stapedius muscle relative to the facial nerve and the stapes.

FIG. 4 shows the location of separate openings in the temporal bone for a stapedius electrode according to an embodiment of the present invention.

FIG. 5 shows the same anatomical locations as in FIG. 4 with the bone removed.

FIG. 6 shows one example of an implantable electrode according to an embodiment of the present invention.

FIG. 7 shows another example of an implantable electrode according to an embodiment of the present invention.

FIGS. 8A-8C shows the distal end structures of an implantable electrode according to various embodiments of the present invention.

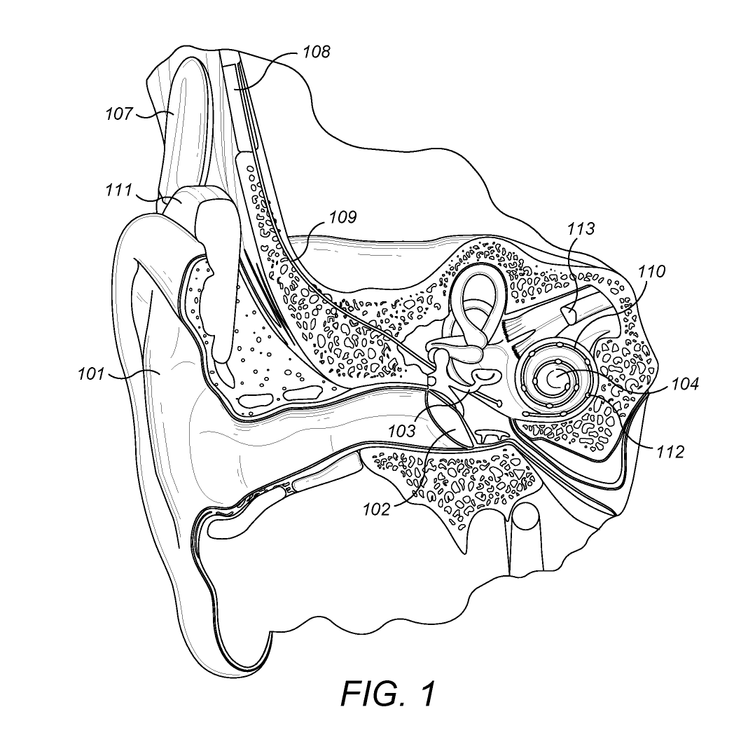

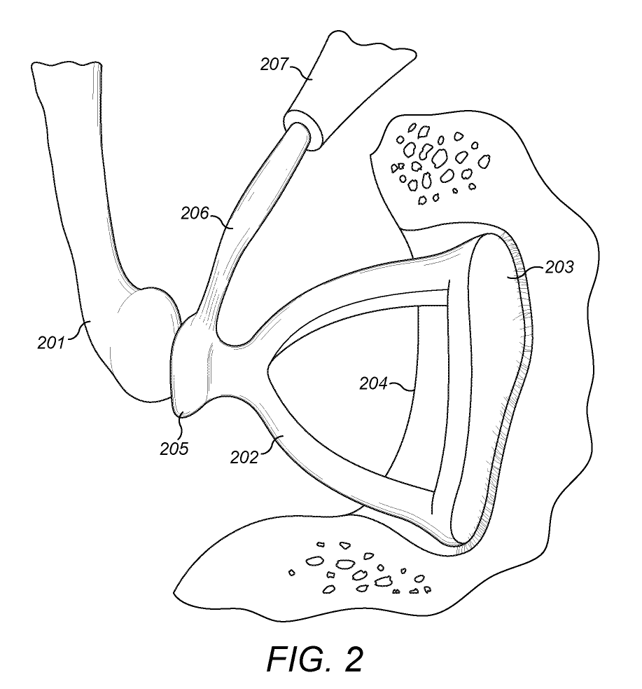

### DETAILED DESCRIPTION

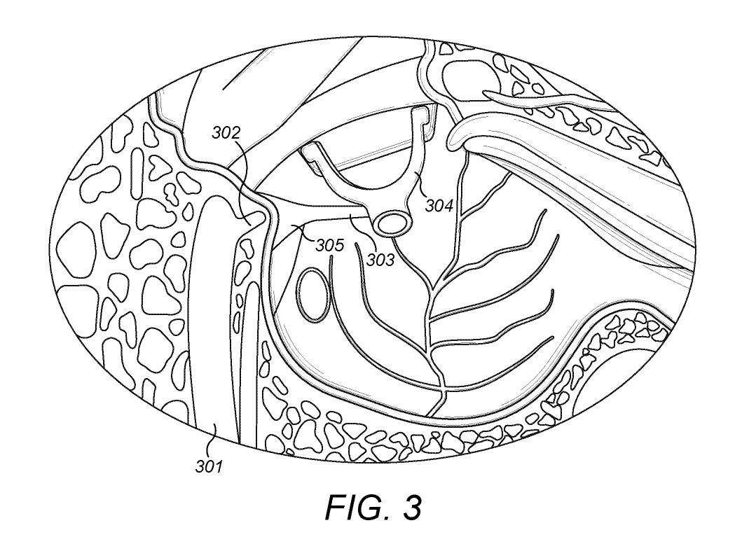

Embodiments of the present invention are directed to methods for inserting a stapedius activity sensor to engage the stapedius muscle from the opposite end to what is conventionally done. FIG. 3 shows the anatomy of the stapedius muscle relative to the facial nerve 301 and the stapes 304. The novel activity sensor is configured to connect to the stapedius muscle belly end 302 near where the facial nerve 301 innervates the stapedius muscle rather than at the tendon end 303 where the stapedius tendon emerges from the pyramidal eminence 305 and connects to the stapes 304. In some patients, the structure of the stapedius muscle, where the facial nerve innervates the stapedius muscle, may not look like a “belly” as such, however, throughout this application, this end is denoted as muscle belly or stapedius muscle belly.

This new approach requires additional surgical drilling, but reaching the belly end 302 of the stapedius muscle will result in a faster, more secure and long term reliable position of the activity sensor within the stapedius muscle, and a more robust, long term measurement signal as compared to placing the activity sensor via the tendon end 303 of the stapedius muscle. This approach is different from prior art methods such as disclosed (for example) in FIGS. 19A-19D of U.S. Pat. No. 6,208,882 where a drilled opening through the pyramidal eminence is suggested, thus creating an access to the stapedius muscle close to where it is connected to the tendon. The improved method described herein is based on the consideration that securing an electrode can be better achieved at the opposite end of the stapedius muscle (i.e. where the nerve innervates the muscle) because the muscle is thicker at this end and consequently a sensor element can be secured better within a larger muscle volume. In addition, the longer bony channel required for the present method automatically secures the corresponding electrode lead better.

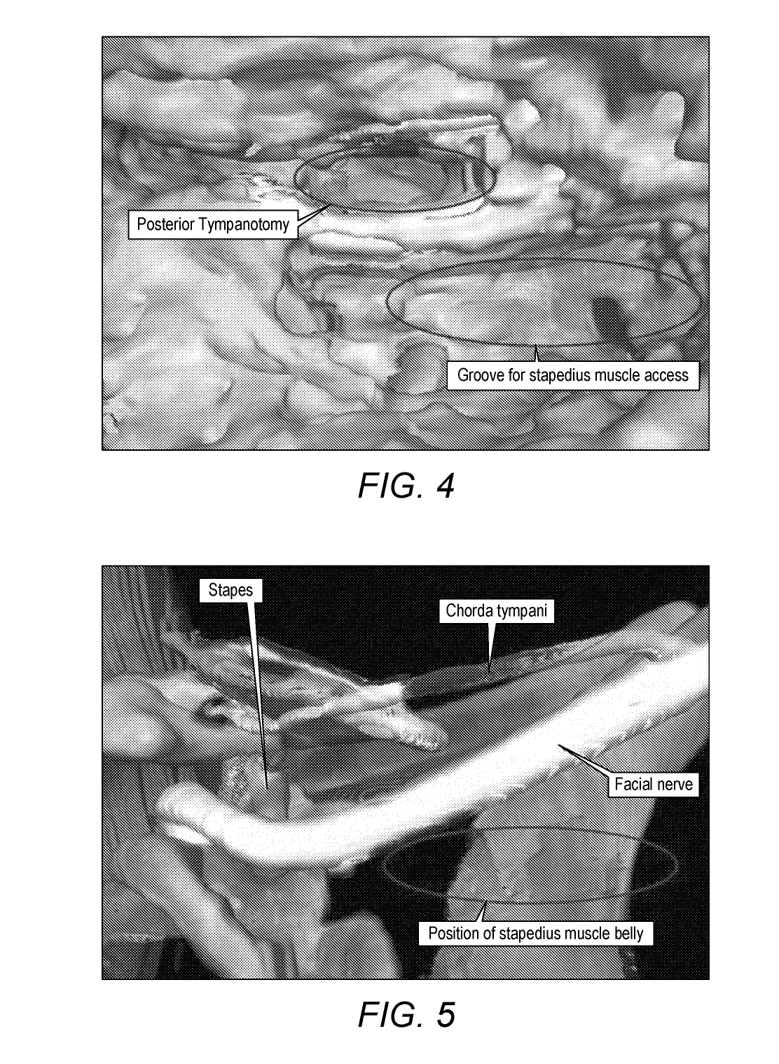

The normal access route for a cochlear implant surgery remains the same via a posterior tympanotomy in the temporal bone, as shown in FIG. 4, that allows finding the anatomical landmarks within the middle ear (see FIG. 5) for a cochleostomy or round window placement of the cochlear electrode array. In an additional step, a sensor lead groove is drilled in the temporal bone at a separate spot that allows access to the belly end of the stapedius muscle near where it is innervated by the facial nerve. After drilling a sufficient posterior tympanotomy opening, the layout of the facial nerve and chorda tympani are then known and a deeper sensor lead groove can be drilled in the temporal bone along the route of the facial nerve (sometimes also called a retro-facialis approach) to reach the stapedius muscle, which is present in the middle between the separation of the chorda tympani and the vestibular semi-circular canals. The final access opening to reach the stapedius muscle can be done by gently drilling or scraping away the final remaining sections of bone to reach the muscle. Once the access opening is created, there are various specific options to place a sensor element into the muscle belly end of the stapedius muscle.

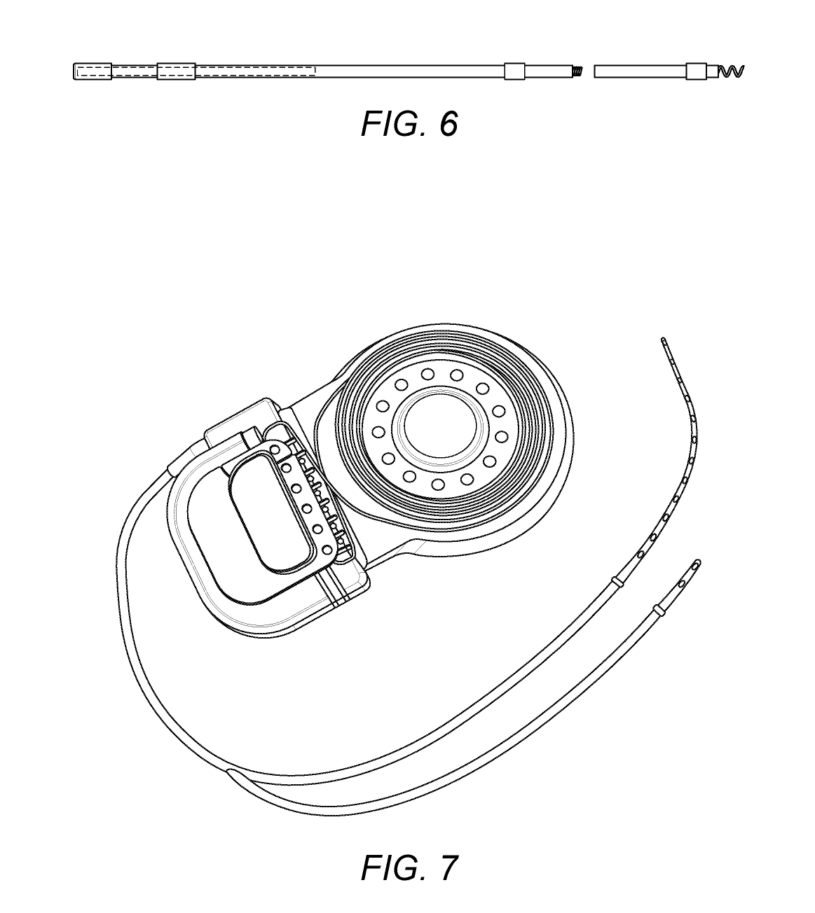

FIG. 6 shows one example of an implantable sensor element in the specific form of a sensing electrode according to an embodiment of the present invention based on a laryngeal pacemaker style electrode with a 3 French electrode contact at the distal end of the electrode lead. From the typical stapedius muscle dimensions reported in the literature (length of 4-5 mm and width of 1.2-2 mm) there will be sufficient space to place a 1 mm diameter 3 French K3P2 laryngeal pacemaker electrode along the belly end of the stapedius muscle. A (bent) insertion tool may be used to place such an electrode in position. The 2½ turns of the electrode contact at the distal end of the electrode lead secures the electrode in a fixed position within the belly end of the stapedius muscle. Thus no additional securing is needed along the electrode lead. In the case of an EMG electrode, a second ring electrode contact is immediately adjacent to the spiral end contact so that bipolar electromyography (EMG) measurement can be taken.

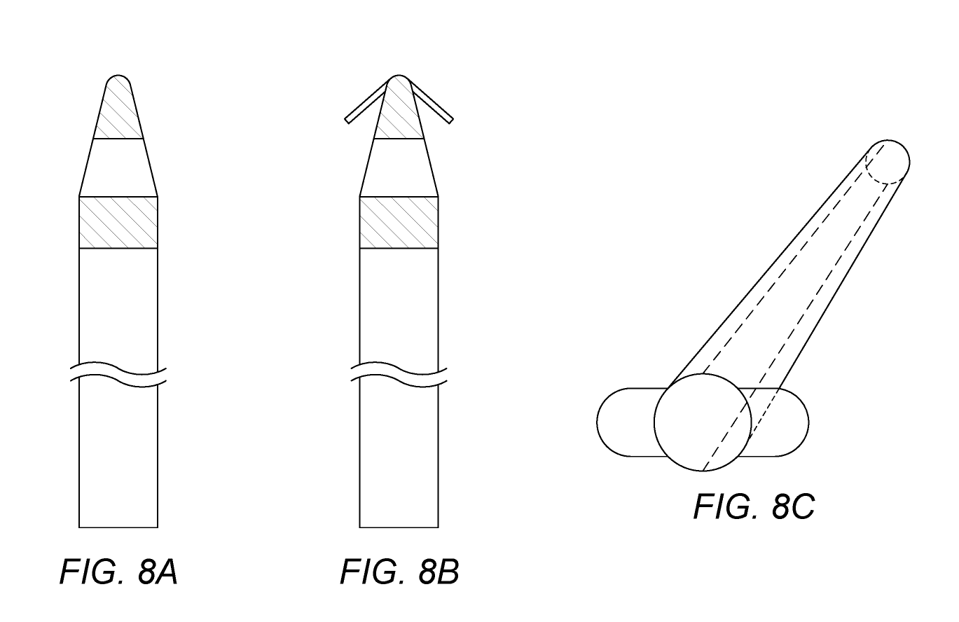

FIG. 7 shows another example of an implantable sensing electrode according to an embodiment having a normal cochlear implant electrode lead with a separate additional sensing electrode lead connected to the implant housing with two electrode contacts at the distal end (see FIG. 8A) for bipolar recording of stapedius muscle EMG signals. This arrangement could be prone to stimulation artifacts arising from capacitive crosstalk when the conductive wires connecting the stimulation sensing/stimulating contacts to the electronic circuitry in the implant housing run parallel to each other in the same electrode lead. In order to avoid this, two separate electrode leads may be provided, one for the wires to the sensing electrode contacts, the other one for the wires to the stimulating electrode contacts.

FIG. 8A shows the distal end of an electrode lead with two electrode contacts for measuring electrical activity of the adjacent stapedius muscle tissue. The electrode tip (around 5 mm to 1 cm) should be more rigid than typical cochlear implant electrodes since more force is needed to insert the electrode into muscle tissue than is needed for insertion of a cochlear implant electrode into the cochlear fluid. The electrode tip also should not be too sharp to avoid damaging the stapedius muscle (with every muscular contraction) and increased growth of fibrous tissue and so a diminished EMG signal over time. An additional fixing element may be used along the length of the electrode lead outside of the stapedius muscle, for example with bone pate or a suture sleeve fixation along the electrode lead. Since the distal end of the electrode lead lacks any fixation structure, muscle contraction could create movement of the electrode relative to the stapedius muscle.

FIG. 8B shows the distal end of an electrode lead having one or more connecting tines (2-4 tines). Such connecting tines can be helpful to secure the electrode contact in a fixed position within the stapedius muscle. Thus no additional fixation elements are needed along the electrode lead, or perhaps only very proximal fixation with bone pate or a suture sleeve. A split cannula as shown in FIG. 8C may be placed around the end of the electrode to allow replacement of the electrode if the initial placement is less than ideal. The split cannula can be inserted into the muscle tissue until the tip and the tines are bent back and no longer hook into the tissue. Then the electrode can be pushed outside the muscle, the tines rebent into their original position, and reinserted into the muscle.

In other specific embodiments, the sensor element specifically may be a pressure sensor configured to measure pressure changes caused by the stapedius muscle during contraction and/or subsequent relaxation. Such a pressure sensor may specifically be a MEMS-based pressure sensor or an optical fiber-based pressure sensor. Using a pressure sensor would avoid a stimulation artifact when this type of stapedius sensor is used in connection with electrical nerve excitation, e.g. with cochlear implants. A combination of EMG and pressure sensor also may be an option to be able combine to small but alternative signals.

This retro-facialis approach may be well suited in combination with robotic surgery techniques. Well-known imaging techniques like computer tomography (CT) scan digital volume tomography (DVT) and/or MRI may be used to record a detailed three dimensional image of the patient's ear anatomy. This image then serves as a patient specific map for the robotic drilling system and/or a presurgical guiding. In particular, after producing the 3D image, a robotic surgical system as known in the art, may drill the access to both the cochlear and the stapedius muscle. The drilled channel can be computed by avoiding critical landmarks such as the semicircular canals, the facial nerve and the corda tympani. Since the stapedius muscle is adjacent to the facial nerve, the bony channel of the facial nerve can be used as a path guide to the muscle. After drilling the channel to the stapedius muscle, a sensor element is inserted. During insertion, the stapedius reflex is continuously elicited (e.g. by electric stimulation via the pre-inserted CI electrode) and the response of the sensor element is recorded. An optimal position of the sensor element adjacent/within the stapedius muscle may be defined by recording a robust reproducible signal (EMG or pressure signal) synchronized with the stimulation signals. When the optimal position is identified, the electrode lead of the sensor element is secured e.g. to bony tissue or to the CI electrode lead to prevent migration over time.

Embodiments of the present invention also include a hearing implant fitting system and/or a hearing implant system (e.g., a cochlear implant, auditory brainstem implant, or middle ear implant) having a device according to any of the foregoing. All these types of electrodes may be operated in a bipolar stimulation mode provided there are two or more contacts on the respective electrode leads directed to the belly of the stapedius muscle. Alternatively, they may be operated in a monopolar stimulation mode with an electrode contact of a cochlear implant electrode or the ground electrode on the implantable housing of the hearing implant as the reference electrode or the reference electrode placed inside the mastoid.

Although various exemplary embodiments of the invention have been disclosed, it should be apparent to those skilled in the art that various changes and modifications can be made which will achieve some of the advantages of the invention without departing from the true scope of the invention.

What is claimed is:

1. A method of placing a stapedius activity sensor in a stapedius muscle of a patient, the stapedius muscle having a tendon end connecting to the stapes bone, and an opposing muscle belly end where the facial nerve innervates the stapedius muscle, the method comprising:
performing a mastoidectomy to create an opening through mastoid bone of the patient;
drilling a lead groove in temporal bone of the patient following a route along the facial nerve to the muscle belly end of the stapedius muscle; and
introducing the stapedius activity sensor along the lead groove to insert a distal end of the stapedius activity sensor into the muscle belly end of the stapedius muscle.

2. The method according to claim 1, further comprising:
implanting a stimulation element for a hearing implant into the patient.

3. The method according to claim 2, wherein the stimulation element is implanted before the stapedius activity sensor is introduced.

4. The method according to claim 2, wherein the stimulation element is implanted after the stapedius activity sensor is introduced.

5. The method according to claim 1, further comprising:
providing test stimulus signals to the facial nerve and the stapedius muscle after drilling the lead groove to confirm proper continuing functioning of the facial nerve and the stapedius muscle.

6. The method according to claim 1, wherein one or more of the steps is performed by robotic surgery.

7. The method according to claim 1, wherein the stapedius activity sensor is a pressure sensor.

8. The method according to claim 1, wherein the stapedius activity sensor is a sensing electrode.

9. The method according to claim 8, wherein the sensing electrode is a 1-3 French diameter electrode.

10. The method according to claim 8, wherein the sensing electrode is an electromyography (EMG) recording electrode.

11. The method according to claim 8, wherein the sensing electrode is a bipolar electrode contact.

12. The method according to claim 8, wherein the sensing electrode includes one or more connecting tines configured to secure the sensing electrode in a fixed position within the stapedius muscle when the distal end of the stapedius activity sensor is inserted into the muscle belly end of the stapedius muscle.
